(19) United States
(12) Patent Application Publication
(10) Pub. No.: US 2014/0257112 A1
(43) Pub. Date: Sep. 11, 2014

(54) GYNECOLOGICAL SCOPE AND MORCELLATION SYSTEMS AND DEVICES

(71) Applicant: Marc Evan Siegel, Alexandria, VA (US)

(72) Inventor: Marc Evan Siegel, Alexandria, VA (US)

(21) Appl. No.: 14/199,796

(22) Filed: Mar. 6, 2014

Related U.S. Application Data
(60) Provisional application No. 61/773,962, filed on Mar. 7, 2013.

(51) Int. Cl. A61B 1/00; A61B 1/04; A61B 1/303; A61B 18/14; A61B 8/12

(52) U.S. Cl. CPC A61B 1/00087; A61B 18/1485; A61B 8/12; A61B 1/303; A61B 1/04; A61B 2017/00367
USPC 600/471; 606/115; 606/110; 606/46

(57) ABSTRACT

An apparatus has an elongate element with a proximal end and a distal end. A connection element is disposed at the proximal end of the elongate element. A morcellator is disposed proximate the distal end. The morcellator has a shank with a base and a leading surface. The base is operably connected to the distal end of the elongate element. The shank at least partially defines a flute disposed substantially helically about an outer surface of the shaft. At least one cutting edge is disposed on the leading surface and proximate the at least one flute.

### CROSS-REFERENCE TO RELATED APPLICATIONS

This application claims priority to and the benefit of U.S. Provisional Patent Application No. 61/773,962, filed Mar. 7, 2013, entitled “GYNECOLOGICAL SCOPE AND MORCELLATION SYSTEMS AND DEVICES,” the disclosure of which is hereby incorporated by reference herein in its entirety.

### BACKGROUND

In the field of gynecology, hysteroscopic surgery is a frequently used modality to treat different pathologies. Performing resections of fibroids, septums, or synechae poses a significant risk of uterine perforation as the operator or surgeon is only able to assess and visualize what is in the cavity with a camera or other device inserted therein. The surgeon often cannot assess the depth of resection within the uterine wall without the use of a trans-abdominal ultrasound.

Hysteroscopic resectors and morcellators often have shortcomings. Often, because of the position of the blade, a morcellator may not be able to approach a fibroid directly from the front of the fibroid. Thus, morcellators are typically configured to resect from the side of the fibroid. This approach restricts the angle of approach to a more difficultly-positioned fibroid. In addition, the cuts made by such morcellators are small and thus are unable to efficiently resect larger myomas before critical fluid deficits are reached. With some resectascopes (e.g., those utilizing wire cautery), a larger myoma can be resected faster. The resectascopes, however, may often have to be removed from the cavity to remove the resection chips, which can obscure the operator's vision.

### SUMMARY

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

In one aspect, the technology relates to an apparatus having: an elongate element having a proximal end and a distal end; a connection element disposed at the proximal end of the elongate element; and a morcellator disposed proximate the distal end, wherein the morcellator includes: a shank having a base and a leading surface, wherein the base is operably connected to the distal end of the elongate element, and wherein the shank at least partially defines a flute disposed substantially helically about an outer surface of the shaft; and at least one cutting edge disposed on the leading surface and proximate the at least one flute. In an embodiment, the shank has an axis; and wherein the leading surface at least partially defines the at least one flute and includes a tip disposed on the axis and a datum plane disposed orthogonal to the axis, wherein the datum plane includes the tip; and wherein the leading surface extends radially from the tip at a surface angle from the datum plane. In another embodiment, the surface angle is between about 10° and about 15°. In yet another embodiment, the base at least partially defines the at least one flute. In still another embodiment, the cutting edge is made of an electrically conductive material.

In another embodiment of the above aspect, a diameter of the elongate element is less than a diameter of the shank. In an embodiment, the morcellator is substantially rigid and the elongate element is flexible relative to the morcellator.

In another aspect, the technology relates to a shank at least partially defining a flute; a base; and a tip disposed opposite the base, wherein the tip has a leading surface at least partially defining a cutting edge, wherein the cutting edge at least partially defines the flute. In an embodiment, an elongate element is adapted to be connected to the base of the morcellator; and a tube defining a lumen and having a distal end and a proximal end, wherein the morcellator is adapted to be rotatably received in the lumen. In another embodiment, a vacuum conduit is disposed proximate the proximal end, wherein the vacuum conduit is adapted to be connected to a source of suction for drawing a fluid from the lumen; and an actuation port disposed proximate the proximal end, wherein the actuation port is adapted to be connected to an actuator disposed external to the cannula, wherein the actuator is adapted to rotatably drive the morcellator. In yet another embodiment, the morcellator is disposed proximate the distal end of the tube. In still another embodiment, an actuator is operatively connected with the elongate element for activating at least one of (a) a rotation of the morcellator, and (b) delivering an electrical current to the cutting edge of the morcellator.

In another embodiment of the above aspect, a pressure sensor for detecting an axial pressure is applied to the morcellator during use. In an embodiment, an image sensing element and an ultrasound emitter are included. In another embodiment, the tube is a non-conductive material.

In another aspect, the technology relates to a cannula having an exterior surface and an interior surface at least partially defining a lumen, wherein the cannula includes a proximal end and a distal end; an ultrasound emitter disposed between the exterior surface and the interior surface; and an image sensor disposed between the exterior surface and the interior surface. In an embodiment, the ultrasound emitter and the image sensor are disposed proximate the distal end. In another embodiment, the cannula further includes: an opening disposed proximate the proximal end, wherein the opening is adapted to be receive on elongate element connected to an actuator disposed external to the cannula, wherein the actuator is adapted to rotatably drive the elongate element disposed within the lumen. In yet another embodiment, a morcellator is connected to the elongate element. In still another embodiment, the morcellator includes: a shank at least partially defining a flute; a base; and a tip disposed opposite the base, wherein the tip has a leading surface at least partially defining a cutting edge, wherein the cutting edge at least partially defines the flute.

### BRIEF DESCRIPTION OF THE DRAWINGS

The same number represents the same element or same type of element in all drawings.

FIG. 1 is a perspective view of an embodiment of a gynecological surgical device.

FIG. 2 is an exploded enlarged partial perspective view of the gynecological surgical device of FIG. 1.

FIG. 3 is an enlarged partial perspective view of the gynecological surgical device of FIG. 1.

FIGS. 4A-4C depict a method of resecting a fibroid, with an embodiment of a gynecological surgical device.

FIG. 5 depicts a perspective view of a dual scope cannula.

FIGS. 6A-6C depict perspective views of the gynecological surgical device of FIG. 5, utilizing a number of accessories.

### DETAILED DESCRIPTION

While the technologies disclosed herein have particular application in the context of gynecological surgery, it will be appreciated that the systems, methods, and apparatuses disclosed can be employed in other types of surgical procedures. For example, the embodiments disclosed herein can be used in minimally invasive surgeries, such as laparoscopic surgeries. Other surgical procedures that would benefit from improved observation capabilities within the surgical cavity or from improved system adaptability may also benefit from the systems disclosed herein. For clarity, however, the technology will be described in the context of gynecological surgical procedures and, more specifically, those procedures that remove fibroids, septa, synechae, or other growths or tissues.

In some embodiments, a resection device configured to accomplish resection of large myomas while removing the tissues and debris during the procedure. In some embodiments, the resector includes a morcellator having a spiral blade that cuts the tissue. A vacuum source draws the resected debris into the flutes of the blade, away from the tip and into a tube so as to remove the debris from the uterine cavity. In some embodiments, at least a portion of the spiral blade is electrified to cauterize while cutting to ensure adequate hemostasis. With the use of a cannula that can include both an image sensor and an ultrasonic emitter (also called a “dual scope cannula”), the surgeon can observe the depth of resection into the uterine wall to prevent perforation and more thoroughly remove the fibroid or tissue. In addition to the resection and cautery capability, in some embodiments, the spiral blade is configured to be enabled when the operator presses down on a starting mechanism and the blade is applied to the area to be resected with a specific amount of pressure. Thus, the blade will not rotate until applied to the area. Additionally, if the blade were to perforate the uterine wall, pressure on the device is lost and rotation of the blade would stop automatically. This helps eliminate or prevent intra-abdominal injury.

In some embodiments, the resection device and tube are configured to be inserted through the dual scope cannula to accomplish various procedures described. Additional accessories or implements can also be utilized with the dual scope cannula to accomplish certain procedures. For instance, an os finder/dilator can be inserted through the cannula for cervical stenosis. The os finder can be inserted into the os and extended using a toggle under direct visualization with the dual scope until the internal os or cavity is reached. Larger dilators can then be attached to safely dilate the cervix. In some embodiments, the dual scope may be used to assess the patency of the Fallopian tubes by observing a distension medium or fluid passing through the tubes. If obstructed, a tubal cannula can be inserted through the dual scope cannula and, under direct view, the ostia can be cannulated and the obstruction relieved. In some embodiments, the dual scope cannula can also be used in the clinical treatment of Asherman's syndrome (uterine synechae). For example, operative scissors can be inserted through the dual scope cannula and be used to lyse the adhesions, with care to minimize further damage to the cavity and prevent perforation.

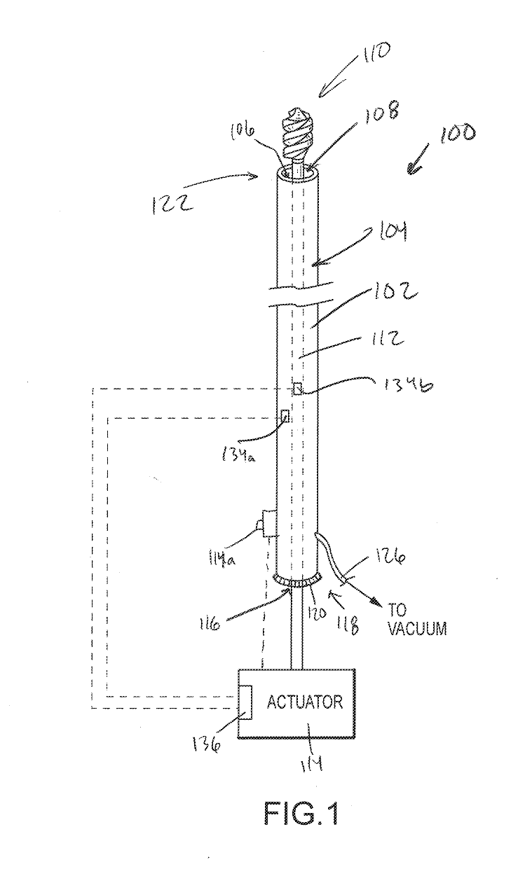

FIG. 1 is a perspective view of an embodiment of a gynecological surgical device 100. The surgical device 100 includes an outer tube 102 defined by a wall having an exterior surface 104 and an interior surface 106. The interior surface defines a lumen 108 in which a morcellator 110 is rotatably disposed for axial movement within the lumen 108. The morcellator 110 is described in more detail below. An elongate element 112 is connected to the morcellator 110 and both rotates and advances the morcellator 110 within the lumen 108. The elongate element 112 can be connected to an actuator 114 which is used to rotate the elongate element 112 (and therefore, the morcellator 110). The actuator 114 may be a powered device, such as an electric motor, or a manually-operated device, such as a foot pedal or hand-crank. A control button 114a may be disposed on the cannula 102 or on the actuator 114 itself. The control button 114a may be incorporated into a handle or may be secured directly to the tube 102 as depicted. The elongate element 112 enters the tube 102 via an actuator port 116, which is disposed proximate the proximal end 118 of the tube 102. In the depicted embodiment, the actuator port 116 is defined by a septum or nipple 120 secured to the proximal end 118, through which the elongate element 112 is passed. The septum 120 forms a seal about the elongate element 112.

The depicted tube 102 also includes a vacuum conduit 126 that connects the lumen 108 of the cannula 102 to a vacuum source. During gynecological surgical procedures, distension fluid is injected into the uterus, so as to expand the cavity. This helps increase visibility within and access to the interior of the uterus. As this fluid is delivered to the uterus, a vacuum is used to draw out excess fluid, as well as tissue, blood, or other debris that is generated by the cutting action of the morcellator.

A sensor 134a may be disposed within the lumen 108 and/or a sensor 134b may be disposed on the elongate element 112. The sensors 134a, 134b may be any type of sensor that is utilized to sense a position of the elongate element 112 within the lumen 108. For example, the sensors 134a, 134b may be a pressure sensor that detects a pressure against the morcellator 110. Alternatively, the sensors 134a, 134b may be a position sensor that detects when the morcellator 110 and/or the elongate element 112 moves axially within the lumen 108. Strain gauges may also be utilized. The sensors 134a, 134b connect to an interlock 136 of the actuator 114, and prevent the actuator 114 from rotating the morcellator 110 unless the morcellator 110 is in contact with a tissue to be removed. Once the interlock 136 determines that the morcellator 110 is in contact with a tissue, typically by a detected movement or pressure, actuation begins to remove said tissue. This safety feature prevents the morcellator 110 from actuating unexpectedly within the uterus, and also disables rotation of the morcellator 110 if the uterus is punctured, or once the target tissue (a septum, for example) has been cut through.

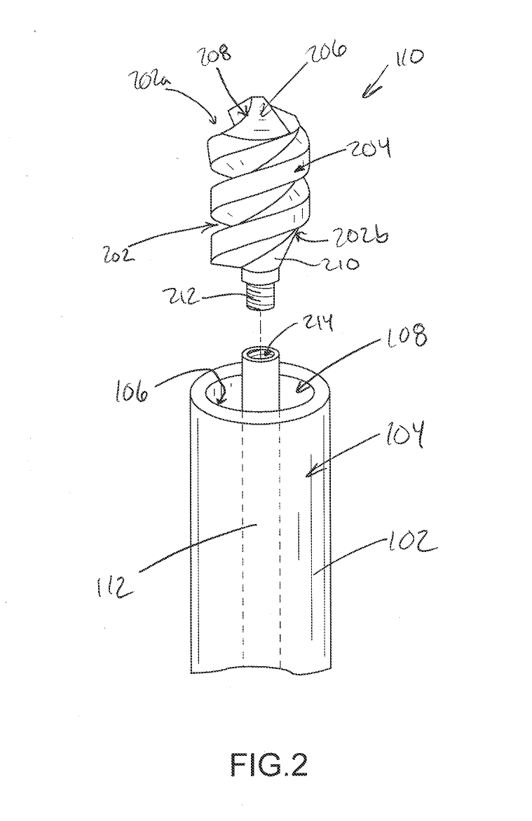

FIG. 2 is an exploded enlarged partial perspective view of the gynecological surgical device 100 of FIG. 1. Elements described above with regard to FIG. 1 are generally not described further. The morcellator 110 is formed from a shank 200 that at least partially defines a flute 202, generally helically formed about an outer surface 204 of the shank 200. The morcellator 110 also includes a leading surface 206. The leading surface 206 at least partially defines a forward portion 202a of the flute 202. A cutting edge 208 is disposed at the leading surface 206 and at least partially defines the flute 202. The flute 202 may also be at least partially defined by a base 210 of the morcellator 110. In the depicted embodiment, the flute 202 has an open rear portion 202b proximate the base 210. This enables smooth flow, via the flute 202, of both fluid and tissue into the lumen 108 of the tube 102, as described in more detail below. The base 210 also includes a connection element 212, in this case a threaded connection, that is adapted to mate with a set of matching threads 214 on the elongate element 112. Other types of connection elements may be used, for example, press-fit connectors, bayonet-style connectors, adhesives, or other types of mechanical fasteners. In other embodiments, the morcellator 110 and elongate element 112 may be a unitary piece.

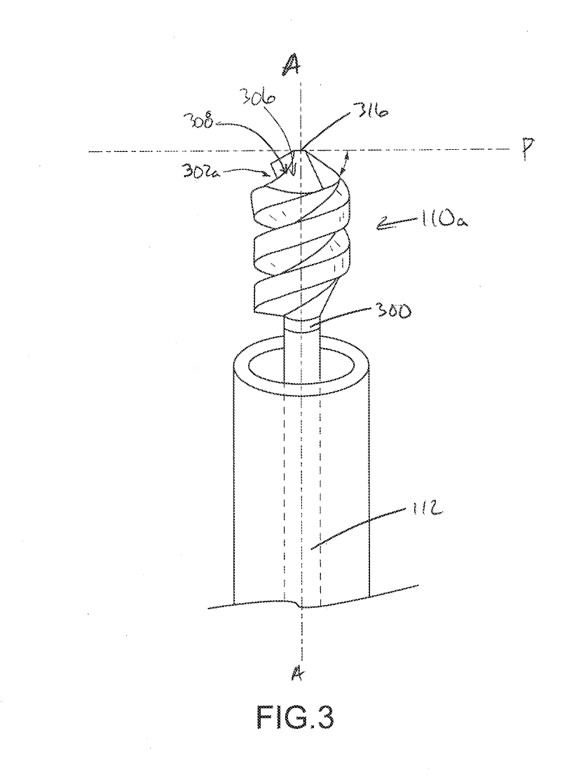

FIG. 3 depicts an enlarged partial perspective view of a morcellator 110a in accordance with an embodiment of the present disclosure. As described above, the leading surface 306 at least partially forms a cutting edge 308 proximate afterward portion 302a of the flute 302. The leading surface 306 terminates at a point or tip 316. An axis A is generally aligned with both a morcellator shank 300 that forms the main body of the morcellator 310a, and the elongate element 112. The tip 316 is also aligned with the axis A. A datum plane P substantially orthogonal to the axis A, and including the tip 316 is also depicted. The leading surface 306 is disposed at an angle α from the datum plane P. The pitch of the angle α may be selected as required or desired for a particular application. In certain embodiments, the angle α may be about 0° to about 40° from the datum plane P. In other embodiments, the angle α may be about 5° to about 35°. In other embodiments, the angle α may be about 10° to about 25°. Other angles are contemplated. The angle α helps assist the tip 316 (and thereafter the leading surface 306 and cutting edge 308) in resecting a target within the uterus.

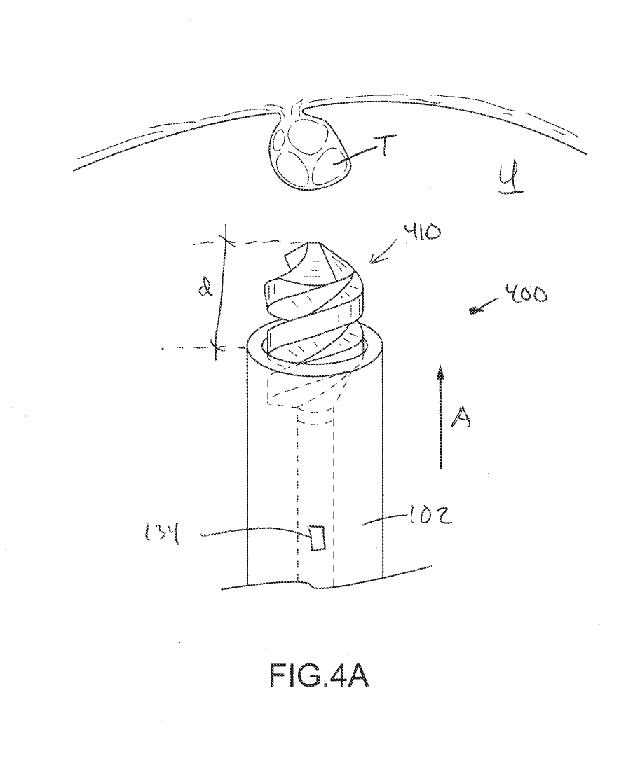

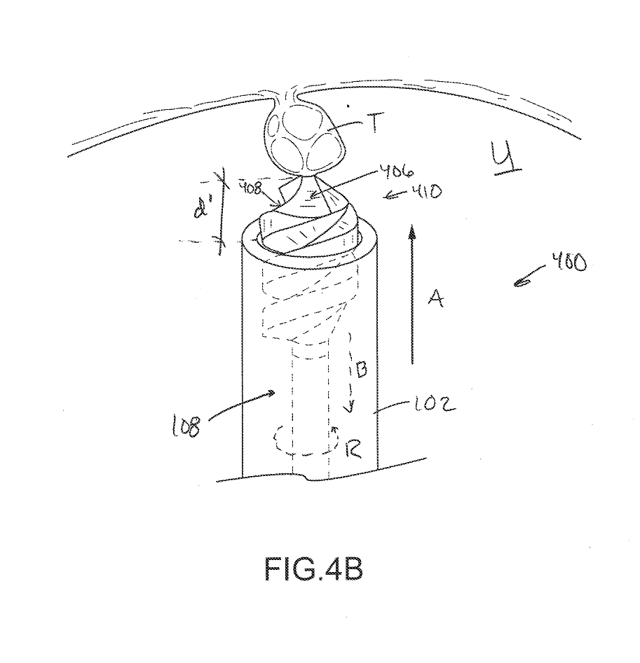

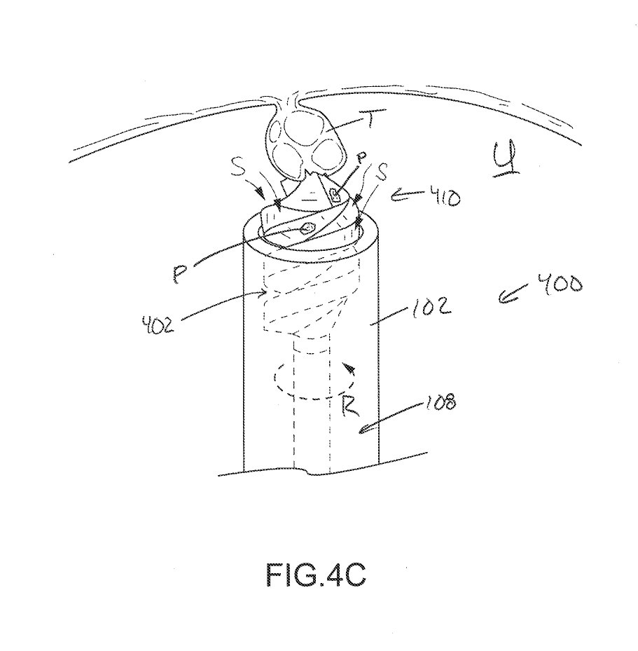

FIGS. 4A-4C depict a method of resecting a target T in a uterus U, with an embodiment of a gynecological surgical device 400. In FIG. 4A, the device 400 inserted into the uterus U and advanced A towards the target T, e.g., a fibroid. In the depicted embodiment, the morcellator 410 extends a distance d out of the tube 102. Once disposed proximate the target T, the surgeon may activate an actuation mechanism (not shown). However, due to the function of the sensor 134 (as described above), rotation of the morcellator 410 cannot begin until the morcellator 410 is in contact with the target T.

In FIG. 4B, the morcellator 410 contacts the target T, which forces the morcellator 410 backwards B, into the lumen 108. Once the morcellator 410 projects a distance d′ from the tube 102, rotation R starts and the cutting edge 408 at the leading surface 406 resects the target T. The actuator may be in electrical communication with the source of vacuum (not shown), such that suction S begins with rotation R. FIG. 4C depicts the morcellator 410 having resected a portion of the target T. Since the vacuum is providing suction S into the lumen 108 of the tube 102, removed portions P of the target T, along with fluid within the uterus U, are drawn into the lumen 108 via the flutes 402. This helps ensure the removed portions P of the target T are removed from the uterus U, to improve visualization of the optical field.

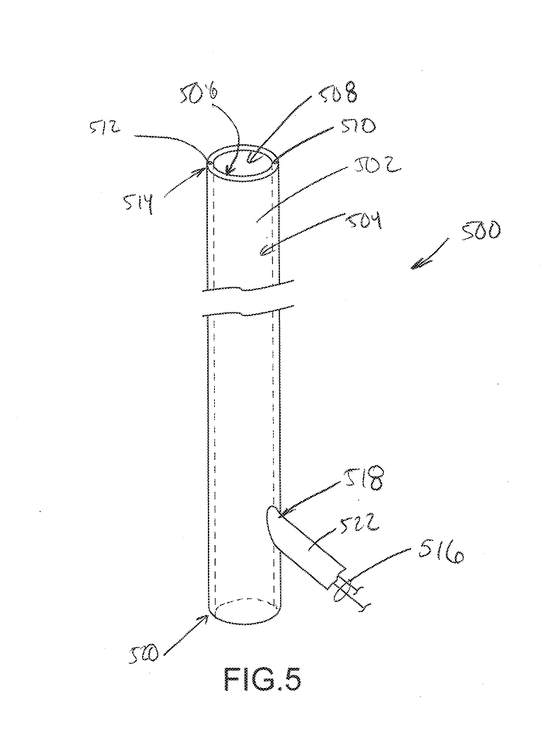

FIG. 5 depicts a perspective view of a dual scope cannula 500. The dual scope cannula 500 is formed from an elongate, tubular wall 502 having an outer surface 504 and an inner surface 506 forming a lumen 508. An ultrasound emitter 510 and an image sensor 512 (such as a microcamera) are disposed proximate a distal end 514 of the tubular wall 502. The depicted embodiment also includes a plurality of wires 516 that exit the tubular wall 502 at a port 518 disposed proximate a distal end 520 thereof. In the depicted embodiment, the wires 516 are contained proximate the tubular wall 502 within a handle 520, which helps manipulate the dual scope cannula 500. These wires 516 are connected to the ultrasound emitter 510 and the image sensor 512. An associated external ultrasound display and an associated image display are connected to the wires 516 to allow the surgeon to view the interior of the uterus during a procedure.

Utilizing both the ultrasound emitter 510 and the image sensor 512, the depicted dual scope cannula 500 obviates the need for a trans-abdominal ultrasound during surgical procedures. In some embodiments, the dual scope cannula 500 provides a surgeon with both an intra-cavitary view and a view through the uterine wall. In the case of a septum resection, a laparoscopy is often needed to assess the contours of the uterus externally, so as to rule out didelphys uterus and prevent perforation. The dual scope cannula 500 can be used to save the expense of a laparoscopy as well as save the patient the need for an abdominal incision. The dual scope cannula 500 can also be used when cervical stenosis is found in a patient. Prior approaches require use of a trans-abdominal ultrasound to guide the surgeon into the external os and up the endocervical canal. This is done to prevent perforation either fundally or laterally. Thus, use of the dual scope cannula 500 could also prevent the formation of pseudo-cavities.

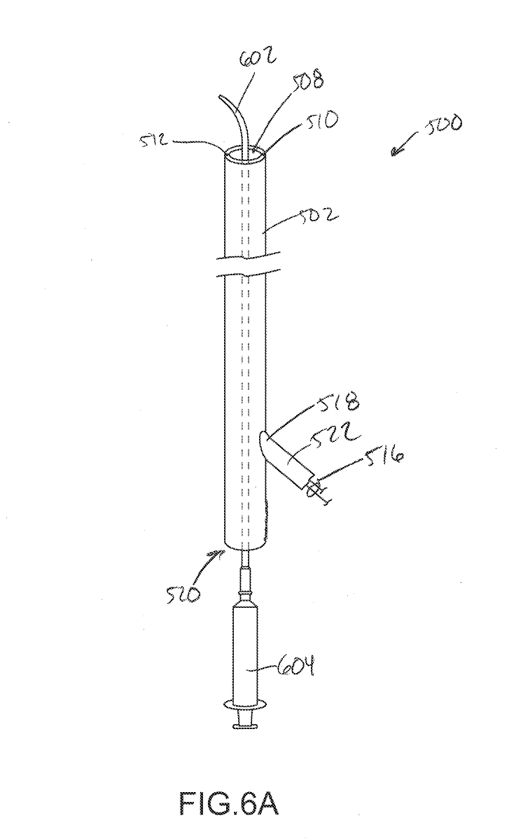

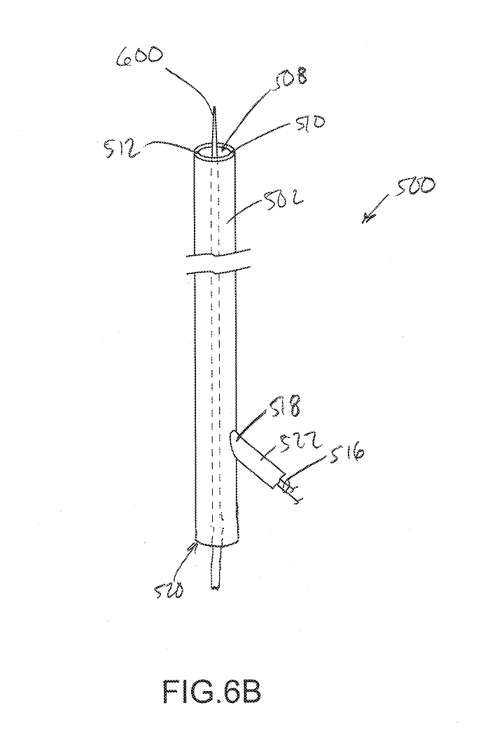

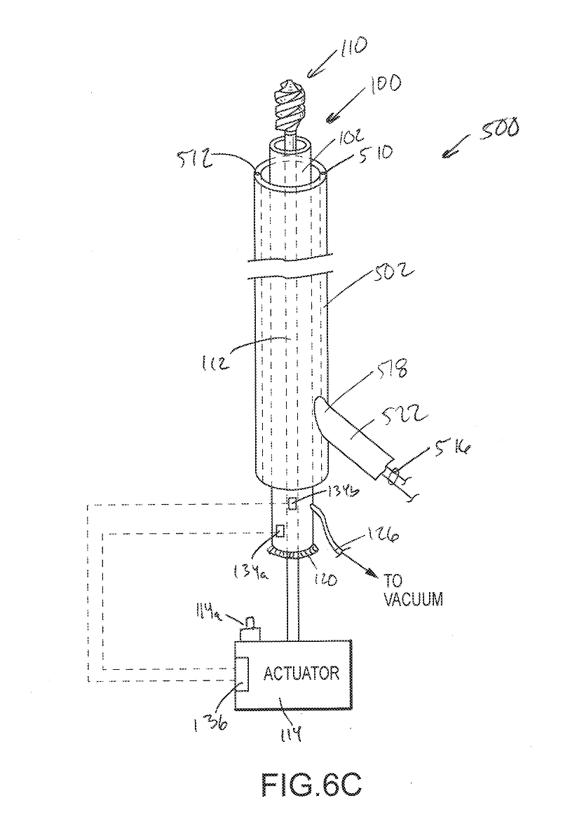

The dual scope cannula 500 depicted herein can also be used in conjunction with other medical devices. For example, FIGS. 6A-6C depict perspective views of other embodiments of the dual scope cannula 500 utilized with accessories. In FIG. 6A, the lumen 508 may receive a smaller tubular cannula 602 for cannulating the ostia with a fluid that can be injected via a syringe 604. In FIG. 6B, the lumen 508 of the dual scope cannula 500 may receive an os finder 600 that is used to dilate the cervix. The surgeon may guide the cannula 500 and os finder 600 (inserted through the proximal end 520) using either or both of the ultrasound emitter 510 and the image sensor 512. FIG. 6C depicts the dual scope cannula 500 used in conjunction with the surgical device 100 described above. Hence, the ultrasound emitter 510 and image sensor 512 can be used to properly position the morcellator 110 during use. In FIG. 6C, the control button 114a is disposed on the actuator 114.

Materials utilized in the manufacture of the surgical device may be those typically used in surgical equipment. Stainless steel, titanium, and other robust metals that may be sterilized may be used. In other applications, aluminum, anodized aluminum, and rigid polymers may be utilized. In some embodiments, the morcellator may include aluminum which has been anodized with a hard coat anodizing process to create an electrical insulated material. The cutting edges of the morcellator may be exposed, i.e., not anodized, such that an electrical charge delivered to the morcellator may be utilized to cauterize the tissue resected by the cutting edge. Carbon fiber-reinforced polymers may be particular useful for the tube or cannula, for example, as they are lightweight, extremely strong, and may be sterilized. Additionally, carbon fiber materials are also non-conductive, and may be particularly useful in embodiments where the morcellator is used for cauterization. Of course, devices utilizing a combination of materials may be used.

Additionally, tubes, cannulas, and elongate elements manufactured of flexible materials allow the surgical devices disclosed herein to approach target disposed in any location within the uterus. Such materials may include flexible but robust plastics, thin flexible metals having memory, or other materials. A device manufactured of such materials is more versatile than devices that could only approach a target tissue from a side of the morcellator.

This disclosure described some embodiments of the present technology with reference to the accompanying drawings, in which only some of the possible embodiments were shown. Other aspects can, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments were described herein, the scope of the technology is not limited to those specific embodiments. One skilled in the art will recognize other embodiments or improvements that are within the scope of the present technology. Therefore, the specific structure, acts, or media are disclosed only as illustrative embodiments. The scope of the technology is defined by the following claims and any equivalents therein.

What is claimed is:

1. An apparatus comprising:
an elongate element comprising a proximal end and a distal end;
a connection element disposed at the proximal end of the elongate element; and
a morcellator disposed proximate the distal end, wherein the morcellator comprises:
a shank comprising a base and a leading surface, wherein the base is operably connected to the distal end of the elongate element, and wherein the shank at least partially defines a flute disposed substantially helically about an outer surface of the shaft; and
at least one cutting edge disposed on the leading surface and proximate the at least one flute.

2. The apparatus of claim 1:
wherein the shank comprises an axis; and
wherein the leading surface at least partially defines the at least one flute and comprises a tip disposed on the axis and a datum plane disposed orthogonal to the axis, wherein the datum plane includes the tip; and
wherein the leading surface extends radially from the tip at a surface angle from the datum plane.

3. The apparatus of claim 2, wherein the surface angle is between about 10° and about 15°.

4. The apparatus of claim 1, wherein the base at least partially defines the at least one flute.

5. The apparatus of claim 1, wherein the cutting edge is comprised of an electrically conductive material.

6. The apparatus of claim 1, wherein a diameter of the elongate element is less than a diameter of the shank.

7. The apparatus of claim 1, wherein the morcellator is substantially rigid and the elongate element is flexible relative to the morcellator.

8. A system comprising:
a morcellator comprising:
a shank at least partially defining a flute;
a base; and
a tip disposed opposite the base, wherein the tip comprises a leading surface at least partially defining a cutting edge, wherein the cutting edge at least partially defines the flute.

9. The system of claim 8, further comprising:
an elongate element adapted to be connected to the base of the morcellator; and
a tube defining a lumen and having a distal end and a proximal end, wherein the morcellator is adapted to be rotatably received in the lumen.

10. The system of claim 9, wherein the cannula defines:
a vacuum conduit disposed proximate the proximal end, wherein the vacuum conduit is adapted to be connected to a source of suction for drawing a fluid from the lumen; and
an actuation port disposed proximate the proximal end, wherein the actuation port is adapted to be connected to an actuator disposed external to the cannula, wherein the actuator is adapted to rotatably drive the morcellator.

11. The system of claim 9, wherein the morcellator is disposed proximate the distal end of the tube.

12. The system of claim 10, further comprising an actuator operatively connected with the elongate element for activating at least one of (a) a rotation of the morcellator, and (b) delivering an electrical current to the cutting edge of the morcellator.

13. The system of claim 12, further comprising a pressure sensor for detecting an axial pressure applied to the morcellator during use.

14. The system of claim 9, further comprising an image sensing element and an ultrasound emitter.

15. The system of claim 12, wherein the tube is comprised of a non-conductive material.

16. A system comprising:
a cannula comprising an exterior surface and an interior surface at least partially defining a lumen, wherein the cannula comprises a proximal end and a distal end;
an ultrasound emitter disposed between the exterior surface and the interior surface; and
an image sensor disposed between the exterior surface and the interior surface.

17. The system of claim 16, wherein the ultrasound emitter and the image sensor are disposed proximate the distal end.

18. The system of claim 16, wherein the cannula further comprises an opening disposed proximate the proximal end, wherein the opening is adapted to be receive on elongate element connected to an actuator disposed external to the cannula, wherein the actuator is adapted to rotatably drive the elongate element disposed within the lumen.

19. The system of claim 18, further comprising a morcellator connected to the elongate element.

20. The system of claim 19, wherein the morcellator comprises:
a shank at least partially defining a flute;
a base; and
a tip disposed opposite the base, wherein the tip comprises a leading surface at least partially defining a cutting edge, wherein the cutting edge at least partially defines the flute.
